(19) United States
(12) Patent Application Publication
(10) Pub. No.: US 2018/0360674 A1
(43) Pub. Date: Dec. 20, 2018

(54) POCKET-SIZED DIAPER-WIPE-PAD KIT

(71) Applicant: Alison Gordon, Atlanta, GA (US)

(72) Inventor: Alison Gordon, Atlanta, GA (US)

(21) Appl. No.: 15/970,156

(22) Filed: May 3, 2018

Related U.S. Application Data
(60) Provisional application No. 62/522,441, filed on Jun. 20, 2017.

(51) Int. Cl. A61F 13/551; A61F 13/49

(52) U.S. Cl. CPC A61F 13/55115; A61F 13/5511; A47D 5/006; A61F 2013/55125; A61F 13/5519; A61F 13/49007

(57) ABSTRACT

A diaper-wipe-pad kit includes a disposable diaper, a packet of at least one disposable wipe, a disposable changing pad and a pouch. The disposable diaper, the packet of at least one disposable wipe and the disposable changing pad are placed in the pouch. The disposable diaper and the disposable changing pad are folded so that the diaper-wipe-pad kit has dimensions that are no greater than 7.5″×5.5″×2″ except for tab portions extending beyond outer seams of the pouch.

### CROSS-REFERENCE TO RELATED APPLICATION(S)

This application claims the benefit of U.S. Provisional Patent Application Serial No. 62/522,441, filed Jun. 20, 2017, the entirety of which is hereby incorporated herein by reference.

### BACKGROUND OF THE INVENTION

### 1. Field of the Invention

The present invention relates to disposable diaper kits and, more specifically, to a diaper-wipe-pad kit having a form factor that fits into a typical back pocket.

### 2. Description of the Related Art

The few diaper-wipe-pad kits that are available currently typically include at least one disposable diaper, a disposable changing pad and a packet of wipes. Sometimes additional items, such as hand sanitizer can be included. Such kits are designed for placement in a diaper changing bag and tend to be large and bulky. Such bulkiness can make it inconvenient when a parent does not want to carry a diaper bag when going somewhere with a baby. Many parents frequently wear trousers with pockets; however, existing diaper kits do not fit comfortably into the pockets.

Therefore, there is a need for a diaper-wipe-pad kit having a form factor that fits into a typical pocket.

### SUMMARY OF THE INVENTION

The disadvantages of the prior art are overcome by the present invention which, in one aspect, is a diaper-wipe-pad kit that includes a disposable diaper, a packet of at least one disposable wipe, a disposable changing pad and a pouch. The disposable diaper, the packet of at least one disposable wipe and the disposable changing pad are placed in the pouch. The disposable diaper and the disposable changing pad are folded so that the diaper-wipe-pad kit has dimensions that are no greater than 7.5″×5.5″×2″ except for seam sealed portions extending beyond outer seams of the pouch.

In another aspect, the diaper-wipe-pad kit has dimensions that are no greater than 7.5″××5.5″×2″ except for seam sealed portions extending beyond outer seams of the pouch and the pouch consists of a moisture proof sheet material.

In yet another aspect, the invention is a method of making a diaper-wipe-pad kit, in which a pouch is made so as to have dimensions that are no greater than 7.5″×5.5″×2″ except for seam sealed portions extending beyond outer seams of the pouch. A disposable diaper is folded so as to have dimensions so that the disposable diaper can fit inside of the pouch and placing the disposable diaper inside of the pouch. A packet of at least one disposable wipe is placed inside of the pouch. A disposable changing pad is placed inside of the pouch. The pouch is sealed after execution of the preceding steps.

These and other aspects of the invention will become apparent from the following description of the preferred embodiments taken in conjunction with the following drawings. As would be obvious to one skilled in the art, many variations and modifications of the invention may be effected without departing from the spirit and scope of the novel concepts of the disclosure.

### BRIEF DESCRIPTION OF THE FIGURES OF THE DRAWINGS

FIG. 1A is a schematic view of a top view of one embodiment of a diaper-wipe-pad kit.

FIG. 1B is a schematic view of a bottom view of the embodiment shown in FIG. 1A.

FIG. 1C is a schematic view of a bottom view of the embodiment shown in FIG. 1A in an opened state.

FIG. 1D is a cross-sectional view of the embodiment shown in FIG. 1A, taken along line 1D-1D.

FIG. 2 is a schematic diagram showing a diaper-wipe-pad kit in use.

FIG. 3A is a schematic view of a top view of one alternative embodiment of a diaper-wipe-pad kit.

FIG. 3B is a schematic view of a bottom view of the embodiment shown in FIG. 3A.

FIG. 3C is a schematic view of a bottom view of the embodiment shown in FIG. 3A in an opened state.

FIG. 3D is a cross-sectional view of the embodiment shown in FIG. 3A, taken along line 3D-3D.

### DETAILED DESCRIPTION OF THE INVENTION

A preferred embodiment of the invention is now described in detail. Referring to the drawings, like numbers indicate like parts throughout the views. Unless otherwise specifically indicated in the disclosure that follows, the drawings are not necessarily drawn to scale. As used in the description herein and throughout the claims, the following terms take the meanings explicitly associated herein, unless the context clearly dictates otherwise: the meaning of “a,” “an,” and “the” includes plural reference, the meaning of “in” includes “in” and “on.”

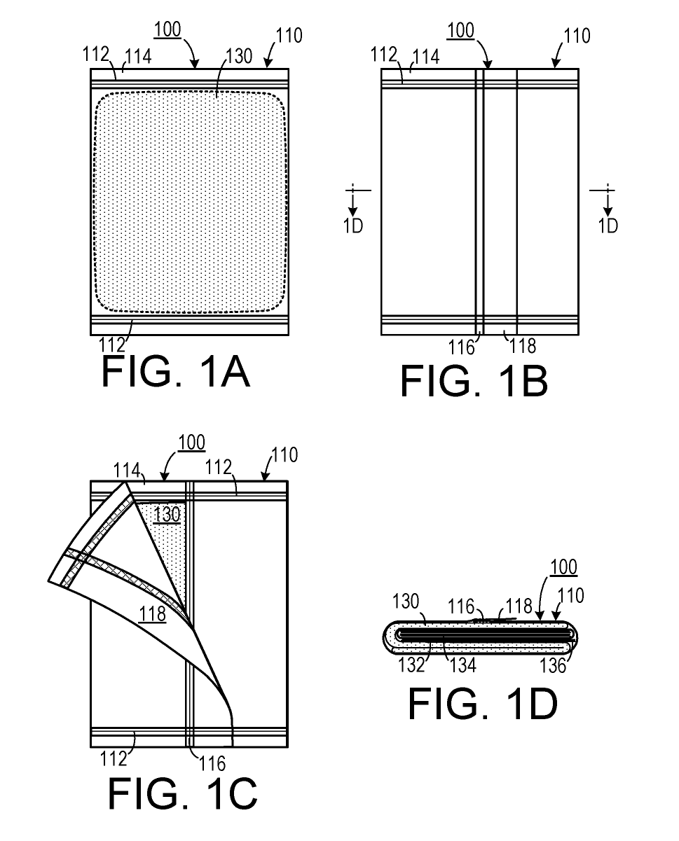

As shown in FIGS. 1A-1D, one embodiment of a diaper-wipe-pad kit 100 includes a pouch 110 (that can include, for example, a flex film, a plastic, biaxially-oriented polyethylene terephthalate—known commercially as “Mylar,” a plastic-lined paper, or one of many other moisture-proof sheet materials) that is sealed around a periphery by two horizontal seams 112, such as welded seams or other types of overwrap seals. In some embodiments, a tab portion 114 can extend beyond the seam 112. A disposable diaper 130, a wipe envelope 132 containing at least one disposable wipe 134 (in the embodiment shown, five wipes 134 are in the wipe packet 132, it will be understood that different numbers of wipes may be included without departing from the scope of the invention) and a disposable changing pad 136 (such as 2-ply paper or plastic-lined paper) are placed within the pouch 110.

Additionally, and informational card can be included in the pouch 110. The card could include marketing information or instructional information printed thereon.

In one embodiment, the pouch can have a tear-away seam flap 118 that extends from a vertical seam 116, thereby allowing access to the contents of the pouch 110. The user can open the pouch 110 by grabbing the flap 118 and pulling it away from the pouch 110, thereby tearing it away from the seams 112 and 116.

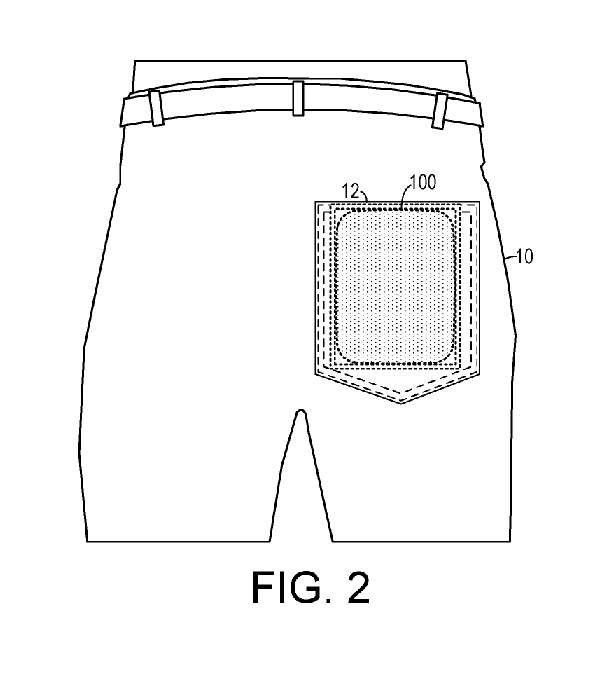

In one embodiment, the diaper 132, the wipe envelope 132 and the plastic-lined paper disposable changing pad 136 are folded and compressed so that they have dimensions that are no greater than 7.5″×5.5″×2″ (in one embodiment the dimensions are no greater than 6″×5″×¾″ and in yet another embodiment, the dimensions are no greater than 6″×3¾″×¾″) except for tab portions 114 extending beyond outer seams 112 of the pouch 110. To achieve this, the diaper 130 may be tri-folded. Thus, as shown in FIG. 2, the entire diaper-wipe-pad kit 100 has dimensions that allow placement in a pocket 12 worn by a user 10.

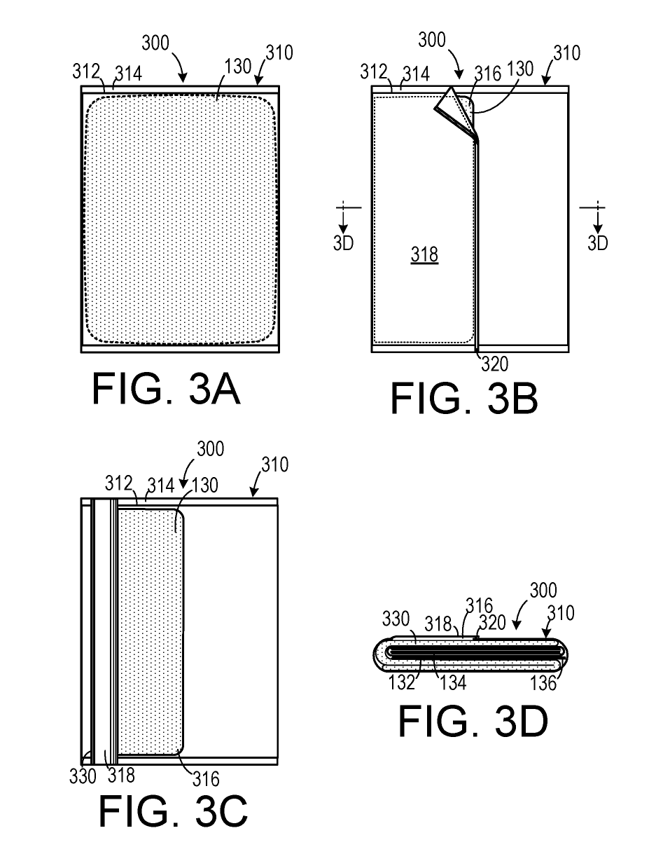

As shown in FIGS. 3A-3D, one embodiment of a diaper-wipe-pad kit 300 includes a pouch 310 that is sealed around a periphery by a seam 312. In some embodiments, a tab portion 314 can extend beyond the seam 312. A disposable diaper 132, a wipe envelope 132 with a resealable access flap containing at least one disposable wipe 134 (in the embodiment shown, five wipes 134 are in the wipe packet 132) and a paper (such as 2-ply paper or plastic-lined paper) disposable changing pad 136 are placed within the pouch 310.

In this embodiment, the pouch can have a resealable seam flap 318 that allows access through an opening 316 to the contents of the pouch 310 and subsequent resealing. In one embodiment, a pressure sensitive adhesive 320 is applied along the flap 318 to facilitate resealing of the pouch 310.

The above described embodiments, while including the preferred embodiment and the best mode of the invention known to the inventor at the time of filing, are given as illustrative examples only. It will be readily appreciated that many deviations may be made from the specific embodiments disclosed in this specification without departing from the spirit and scope of the invention. Accordingly, the scope of the invention is to be determined by the claims below rather than being limited to the specifically described embodiments above.

What is claimed is:

1. A diaper-wipe-pad kit, comprising:
(a) a disposable diaper;
(b) a packet of at least one disposable wipe;
(c) a disposable changing pad; and
(d) an pouch into which is disposed the disposable diaper, the packet of at least one disposable wipe and the disposable changing pad, wherein the disposable diaper and the disposable changing pad are folded so that the diaper-wipe-pad kit has dimensions that are no greater than 7.5″×5.5″×2″ except for tab portions extending beyond outer seams of the pouch.

2. The diaper-wipe-pad kit of claim 1, wherein the pouch comprises a moisture proof sheet material.

3. The diaper-wipe-pad kit of claim 1, wherein the packet of at least one disposable wipe includes five disposable wipes.

4. The diaper-wipe-pad kit of claim 3, the packet comprises an envelope into which the wipes are disposed and wherein the envelope includes a resealable access flap that, when opened, allows access to the wipes.

5. The diaper-wipe-pad kit of claim 1, wherein the diaper is a multi-folded diaper.

6. The diaper-wipe-pad kit of claim 1, wherein the disposable changing pad includes a paper configured to serve as a protective barrier during a changing process.

6. The diaper-wipe-pad kit of claim 6, wherein the paper includes a 2-ply paper.

8. The diaper-wipe-pad kit of claim 6, wherein the paper includes a plastic-lined paper.

9. A diaper-wipe-pad unit, comprising:
(a) a disposable diaper;
(b) a packet of at least one disposable wipe;
(c) a disposable changing pad; and
(d) an pouch into which is disposed the disposable diaper, the packet of at least one disposable wipe and the disposable changing pad, wherein the disposable diaper and the disposable changing pad are folded so that the diaper-wipe-pad kit has dimensions that are no greater than 7.5″×5.5″×2″ except for tab portions extending beyond outer seams of the pouch, the pouch consisting of a moisture proof sheet material.

10. The diaper-wipe-pad unit of claim 9, wherein the packet of at least one disposable wipe includes five disposable wipes.

11. The diaper-wipe-pad unit of claim 10, the packet comprises an envelope into which the wipes are disposed and wherein the envelope includes a resealable access flap that, when opened, allows access to the wipes.

12. The diaper-wipe-pad unit of claim 9, wherein the diaper is a multi-folded diaper.

13. The diaper-wipe-pad unit of claim 9, wherein the disposable changing pad includes a paper configured to serve as a protective barrier during a changing process.

14. The diaper-wipe-pad kit of claim 13, wherein the paper includes a 2-ply paper.

15. The diaper-wipe-pad kit of claim 13, wherein the paper includes a plastic-lined paper.

16. A method of making a diaper-wipe-pad kit, comprising the steps of:
(a) making a pouch so as to have dimensions that are no greater than 7.5″×5.5″×2″ except for tab portions extending beyond outer seams of the pouch;
(b) folding a disposable diaper so as to have dimensions so that the disposable diaper can fit inside of the pouch and placing the disposable diaper inside of the pouch;
(c) placing a packet of at least one disposable wipe inside of the pouch;
(d) placing a disposable changing pad inside of the pouch; and
(e) sealing the pouch after execution of the preceding steps.

17. The method of claim 16, wherein the step of folding the disposable diaper comprises multi-folding the diaper.

18. The method of claim 16, wherein the step of making a pouch is performed so that the pouch has dimensions that are no greater than 6″×5″×¾″.

19. The method of claim 16, wherein the pouch consists of a moisture proof sheet material.
